(19) United States
(12) Patent Application Publication
(10) Pub. No.: US 2024/0210382 A1
(43) Pub. Date: Jun. 27, 2024

(54) METHODS, DATABASE AND SYSTEM FOR USE WITH VIRAL INFECTION

(71) Applicant: RESPIRATION SCAN LTD, Ness Ziona (IL)

(72) Inventors: Yaron Lapidot, Givat Ada (IL); Igal Moshe Bar Ilan, Hevel Korazim (IL)

(21) Appl. No.: 17/911,519

(22) PCT Filed: Mar. 18, 2021
(86) PCT No.: PCT/IL2021/050297

§ 371 Date: Sep. 14, 2022

Related U.S. Application Data
(60) Provisional application No. 62/991,664, filed on Mar. 19, 2020.

(51) Int. Cl. G01N 33/497; G16H 10/40; G16H 50/20

(52) U.S. Cl. CPC G01N 33/497; G16H 10/40; G16H 50/20; G01N 2033/4975

(57) ABSTRACT

The invention disclosed herein concerns screening and detecting compounds associated with viral replication and use thereof for early detection of a variety of viral infections.

### TECHNOLOGICAL FIELD

The present disclosure concerns methods relating to viral infection.

### BACKGROUND ART

References considered to be relevant as background to the presently disclosed subject matter are listed below:
International Patent Application Publication No. WO2021/028928

Acknowledgement of the above reference herein is not to be inferred as meaning that these are in any way relevant to the patentability of the presently disclosed subject matter.

### BACKGROUND

WO2021/028928 describes screening and early detection of a variety of disease conditions based on the identification of at least one disease-associated marker in a breath sample from a subject. Specifically, there is disclosed a method that comprises exposing to a breath sample a sampling unit comprising an adsorbing region capable of reversibly associating volatiles in the breath sample, analyzing the sampling unit to identify volatiles adsorbed onto the adsorbing regions and determining presence of at least one disease-associated marker, wherein an increase or a decrease in an amount of said marker as compared to an amount of said marker measured at an earlier time point being indicative of existence of a disease state. Likewise, the appearance or disappearance of a marker detected earlier may also provide an indication on disease state.

### GENERAL DESCRIPTION

The present disclosure is based on the development of a unique methodology that relies on the identification of volatile organic compounds (VOCs) and semi-volatile compounds (sVOCs) (collectively referred to herein as “VOCs”) released from viral infected cells during replication of the virus therein, and the use of the outcome of this unique methodology for detection of viral infections, including identifying the type of virus to thereby enable early intervention and treatment. Once the VOCs have been identified in association with a specific viral infection, they can be from a signatory viral marker profile for a specific virus/viral infection, and this signatory viral marker profile can be identified from exhaled breath samples of individuals suspected of being infected or identified as being infected by a virus.

The present disclosure is also based on the experimental demonstration that different viruses are characterized by different VOCs, e.g. volatile metabolites or collection of such VOCs. Such collection of VOCs is thus used, in the context of the present disclosure, in various methods involved in the identification, monitoring and/or treatment of viral infection.

The identification of signatory markers profile for different viruses can then be used to establish a database of such signatory markers profile, which can be utilized in various diagnostic and medical methods.

In accordance with a first of its aspects, the present disclosure thus provides a method of determining viral infection in a subject, the method comprises analyzing a breath sample of said subject, said analysis comprises:
a. from said breath sample, obtaining input data specifying one or more volatile organic compounds (VOC) associated with replication of a defined virus in a host cell;
b. querying a database using said input data to identify a marker profile associated with the at least one of said specified one or more VOC; and
c. contingent upon the identification of said marker profile in said database, providing output data comprising said marker profile and the defined virus.

In accordance with a second aspect, the present disclosure provides a method for monitoring a viral infection in a subject, the method comprises:
a. obtaining from a breath sample of said subject input data specifying one or more volatile organic compounds (VOC), said sample being obtained at a determination time point;
b. querying a database using said input data to identify a marker profile associated with the at least one of said specified one or more VOC;
c. contingent upon the identification of said specified marker profile in said database, providing output data comprising said marker profile associated with the defined virus; and
d. comparing said output data with prior data corresponding to a marker profile obtained for said subject at a time preceding the determination time point; wherein a change in said marker profile being indicative of a viral infection state.

In accordance with a third of its aspects, the present disclosure provides a method of determining a treatment protocol for a subject being suspected of having a viral infection, the method comprises:
a. determining for said subject a viral marker profile, said determination comprises:
   i. from said breath sample obtaining input data specifying one or more volatile organic compounds (VOC) associated with replication of a defined virus in a host cell;
   ii. querying a database using said input data to identify said viral marker profile, the marker profile being associated in said database with a defined virus and a defined host cell; and
   iii. contingent upon identification of said viral marker profile, providing output data with respect to the defined virus and the defined host cell; said output data comprises determination of type of virus causing said viral infection in said subject;
b. determining or selecting a treatment protocol for the identified virus.

Yet, in accordance with a fourth aspect, the present disclosure provides a method of treatment of a subject having a viral infection, the method comprises providing said subject with an anti-viral treatment, wherein said anti-viral treatment is determined by the method of determining viral infection as disclosed herein.

Further, the present disclosure provides, in accordance with its fifth aspect, a database for use in any of the disclosed methods, the database comprises a structured collection of records including a bank of marker profiles associated with a bank of defined viruses, wherein said viral marker profile comprises specified VOCs that are released from at least one defined host cell, during viral replication with a defined virus.

Finally, the present disclosure provides a system comprising:
a. an input interface for receiving input data specifying one or more VOCs;
b. a management module comprising a database responsive to a query specifying the one or more VOCs for returning a respective marker profile;
c. an output provisioning module adapted to provide as output data any one or combination of a defined virus and a defined host cell; each being associated with a marker profile; wherein said database comprises a structured collection of records including a bank of marker profiles associated with a bank of defined viruses, and optionally with a bank of defined host cells, wherein each of said marker profile comprises specified VOCs that are released from at least one defined host cell, during viral replication with the defined virus.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B provides images of two possible in vitro systems for identification of volatile organic compounds released from a culture media during viral replication.

FIGS. 2A-2B are bar graphs showing (correlated) amounts of Tridecane (FIG. 2A) and 1,4 dichloro-benzene (FIG. 2B) released from A549 cells infected by 229E coronavirus as a function of time from inoculation.

FIGS. 3A-3C are bar graphs showing (correlated) amounts of 2,2,4,6,6-pentamethyl-heptan (FIG. 3A), 2-ethyl-oxetane (FIG. 3B) and Logifolene ((1R,2S,7S,9S)-3,3,7-trimethyl-8-methylenetricyclo-[5.4.0.0^{2,9}]undecane) (FIG. 3C) released from HCT8, MRC5 and A549 cells, respectively, infected by OC43 coronavirus as a function of time from inoculation.

FIGS. 4A-4B are bar graphs showing (correlated) amounts of heptadecyl ester of 2-fluorobenzoic acid (FIG. 4A) and bis-trimethylsilyl ether of 2,3-dimethylhydroquinone (FIG. 4B) released from A549 cells infected with H1N1 Influenza A virus as a function of time from inoculation.

FIGS. 5A-5G are microscope images of Vero cells inoculated with SARS CoV2 (wild type) at inoculation time (t=0) (FIGS. 5A, 5B), after 24 hours (FIG. 5C, 5D, 5E) or 48 hours (FIG. 5F, 5G), and after 72 hours (FIG. 5H, 5I, 5J, 5K).

### DETAILED DESCRIPTION OF EMBODIMENTS

Compounds present in the exhaled breath provide an indication on the mammals' health and can possibly provide an early diagnosis or improved management of diseases. When aiming at management of viral infections, the situation may somewhat be different from other pathogen-induced infections, i.e., for example bacteria, where it has been found by the present inventors that the compounds present in the exhaled breath are of virus origin per se and/or the host cell in which the virus replicates.

Generally, a virus is a non-living parasite that requires a target host cell for its replication. Through the generation of abundant copies of its genome and packaging these copies, the virus continues infecting new host cells. The result of viral replication in the host cell invariably results in the destruction of the host cell and as a result, cytoplasmatic elements of the target host cell are released into the surrounding. Within the host cells' debris are remains of cytoplasmatic cellular metabolites that were produced during replication of the virus and metabolites originating from the virus. Since the process of viral replication is a precise repeatable process, the cell debris of a specific host cell infected with a specific virus is expected to be repeatable and also precise in nature.

Therefore, it has been envisaged that identification of volatile metabolites of cell debris indicate on and can identify the type of virus that has invaded the cell. Firstly, each virus has its own characteristic metabolites when it replicates. Secondly, specific cell debris of a specific host cell infected with a specific virus can also be used as a fingerprint/biomarker for a specific viral infection.

Further, it has now been found that cellular debris from viral replicating cells, contains volatile organic compounds (VOCs) and semi-volatile organic compounds (sVOCs), which are unique to the virus itself and to the virus within defined host cell(s). Thus, the collection of such host/virus specific VOCs and/or sVOCs define a compounds' profile or a viral signatory marker profile for a specific viral infection, with optionally the association to a specific host cell of which it has infected.

It has thus been realized that the identification of VOCs and/or sVOCs released from the debris of viral replicating cells (as a result of the cells' lysis), using any ex vivo assay, e.g. using in vitro cultures, can be used to construct a unique database of virus/host specific marker profiles as further defined hereinbelow, and use this database for management of viral infections at different disease stages.

Specifically, the above realization may be beneficial in the detection and/or management of viral infection in the respiratory tract, e.g., infections caused by viruses that harbor target cells on the lining of the respiratory tract. For example, the coronavirus connects to a host cell in the epithelial lining of the nasal cavity and pharynx to enter and proceed with replication. Once replication is completed at least some VOC's and/or SVOCs of the epithelial cell debris are exhaled with the breath.

The above realization is also supported by experimental data showing that for at least common coronaviruses, wild type (lethal) coronavirus and the influenza A N1H1 virus it was possible to identify, with high probability, unique viral marker profiles when these viruses were inoculated within specific host cells.

Thus, the present disclosure provides, methods and system that utilize a database, also forming part of the present disclosure, constructed based on the in vitro identification of one or more VOCs and sVOCs released to the surrounding of viral infected cellular debris and are directly associated with the viral replication and hence infection.

For the sake of simplicity, when referring to VOCs it is to be understood to encompass VOCs or sVOCs. The term VOC and VOCs are used interchangeably.

With respect to VOCs, it is to be understood that the compounds in surrounding of viral infected cellular debris are the compounds released to the gaseous environment surrounding the medium holding the debris (e.g. to a space above or in proximity to the medium); and/or in sputum, i.e., liquid medium holding the debris.

For establishing a database of the unique VOCs that are specific to a virus and thus form the viral signatory marker profiles there is a need to distinguish between release compounds arising from the virus replication and from the debris of viral replicating cells (as a result of the cells' lysis) and background volatile compounds, i.e., compounds arising from the cell irrespective of the presence of the virus, e.g. prior to the replication of the virus, and/or compounds released from the growth medium, and/or even compounds released from the material forming the apparatus accommodating the cells/medium/virus. Such background compounds are considered as “noise” and are all subtracted, as further discussed below.

In some cases, the background VOCs are VOCs that are common to more than one virus. In other words, to increase the specificity to a defined virus, in case there are identified VOCs that are common/shared by two or more viruses, these common/shared VOCs are not considered in the identification of a virus.

Based on the identification of unique VOCs that are specific to a virus and the specific host cell of which it has replicated or to a virus, a database is constructed, and this database can be used for various determination methods. The database can be continuously updated with new “clusters” of specified VOCs associated with a defined virus and optionally with a defined host cell. For example, once a virus, whether a known virus, that has been isolated, any of its mutations, or a newly identified virus (e.g. mutation of an existing virus) is isolated, its characteristic VOCs can be determined by a simple in vitro method (such as that described hereinbelow), and the specified VOCs can then be added to the database and used for swift and quick identification of the existence of the virus in an individual.

Since the database includes clusters of VOCs associated with defined viruses and defined host cells, the database can best be used to identify a viral infection or the state of viral infection from breath samples of individuals. Thus, according to its broadest aspect, the present disclosure provides methods comprising analyzing a breath sample of a subject. Such methods involve, as an initial step, obtaining input data specifying one or more VOCs from one or more breath samples of a subject; querying a database using the input data to identify a signatory viral marker profile, the marker profile being associated in the databank with a defined virus and optionally with a defined host cell; and contingent upon the identification of the marker profile in the database, providing output data with respect to the defined virus and optionally the defined host cell both a priori associated with said marker profile. The data can comprise, inter alia, determination of type of viral infection in the subject, and type of host cell or tissue or organ being infected by the virus.

In the context of the present disclosure, when referring to “viral infection” it is to be understood as encompassing any condition that involves the harboring of host cells by a replicating virus. For the purpose of the present disclosure, the viral infection necessarily also involves the replication of the virus within the host cells.

The viral infection can correspond to an infection by different viruses within a family, such as coronaviridae, to different viruses within a group, such as the group of coronaviruses, as well as to an infection by a specific virus strain. In one particular example, when referring to a viral infection it is to be understood to be strain specific.

In the context of the present disclosure, when referring to “determining viral infection” it is to be understood as encompassing any determination relating to a state of a viral infection, including, inter alia, any one of onset of the infection, level of infection, level of progression/regression of the infection, etc.

In some examples, the viral infection comprises or is an infection along a part/organ/segment of the respiratory tract.

In some examples, the viral infection comprises or is an infection along the upper respiratory tract.

In some examples, the viral infection comprises infection of epithelial cells lining the nasal cavity and/or pharynx.

A non-limiting list of viral infections that fall within the scope of the present disclosure include those caused by a virus from any one of the following virus families: Coronaviridea, Adenoviruses, Orthomyxoviruses, Picornaviruses and Rhinoviruses. These are known to cause disease in the upper respiratory tract.

In some examples, the Arboviruses, Arenaviruses, Paramyxoviruses, Poxviruses and Retroviruses virus families are included. These are transmitted through the airways and mainly manifest in a different clinical syndrome but sometimes there is a manifestation of upper airways.

In some examples, the virus is selected from viruses that infect humans through zoonosis such as SARS-CoV-2. These viruses may not have yet been manifested in human upper respiratory airways. However, their appearance is statistically certain, and their appearance and effect fall within the scope of the present disclosure.

In some examples, the virus is an enveloped virus. Examples of enveloped viruses include, without being limited thereto, influenza virus and coronavirus.

When referring to coronavirus, it may include any one of SARS-CoV-2, SARS-CoV-19 (Covid-19 disease), MERS-CoV (MERS disease), SARS-CoV (SARS disease), as well as the more common human coronavirus strains known as Corona 229E, ML63, OC43 and HKU1.

In some examples, the virus is a non-enveloped virus, such as norovirus or parvovirus.

The herein disclosed method utilized a priori analysis of a subject's breath.

The “breath sample” is a sample obtained actively or passively from the subject's exhaled breath.

Passive sampling involves capturing of the volatiles and/or semi-volatiles without applying any specific physical intervention. An example of passive sampling is a sampling unit that is equipped with an opening designed for Venturi effect.

Active sampling involves capturing the volatiles and/or semi volatiles through the implementation of, e.g., a pump (mechanical, electric, Helium or other) or a suction unit.

Typically, samples are collected directly into sampling units and the volatiles and/or semi-volatiles are allowed to interact by adsorption to one or more adsorbing regions. Where the subject's full cooperation is provided, the subject exhales into the sampling units and the samples are thereafter processed. Where cooperation of the subject is not possible, samples may be collected from the subject's oral cavity or from the lungs. Samples from subjects on ventilation machines may be obtained by associating the sampling units to the outlet line of the respiration unit, as disclosed herein.

In some examples, the method comprises obtaining a breath sample from a subject by employing any non-invasive means known in the art. Non-limiting methods for collecting exhaled breath samples may involve the use of apparatuses approved by the American Thoracic Society/European Respiratory Society (ATS/ERS), see for example Silkoff et al., Am. J. Respir. Crit. Care Med., 2005, 171, 912.

In some examples, the sample may be obtained by direct exhalation of breath into a measuring device or apparatus, such as the desorption tool described in WO2021/028928, the content of which is incorporated herein by reference in its entirety.

Briefly, exhaled breath is collected by an adsorbing/sampling unit(s).

A “sampling unit” can be any means for collecting. In some examples, the sampling unit is a container or a vessel or a canister of any shape or size configured for receiving and holding a breath sample, or liquid solution exhaled with the breath containing volatiles, via dedicated adsorbing regions, each being capable of reversibly associating to the exhaled compounds. To this end, adsorbing regions are of a material that is configured to physically trap the volatile and/or semi volatile compounds. The adsorbing region materials can be characterized by presence of surface pores, surface roughness, increased surface area, and the like. Notwithstanding the structural characteristics, the adsorptive surface or material can be tailored for selective adsorption, as disclosed herein, or a non-selective adsorption.

In some examples, the adsorbing regions are formed of a material selected from organic porous polymers such as poly(2,6-diphenyl-p-phenylene oxide (PPPO), sulfonated polymers, ion-exchange resins, carbon molecular sieves that are prepared by controlled pyrolysis of poly(vinylidene chloride) or sulfonated polymers, and others. A non-limited list of possible materials for use in the adsorbing region is provided in WO2021/028928, the content of which is incorporated herein by reference in its entirety.

In some examples, the adsorbing region comprise metal-based materials, such as, without being limited thereto, metal nanoparticles of any sort, metallic surfaces, metal matrices and others.

In some examples, the adsorbing region is designed with adsorbents that have a surface area between 5 and 1,500 m²/g, a density of between 0.2 and 0.7 and/or a micropore diameter between 4 and 300 Å.

Exposure of a sampling unit to a subject's breath can be achievable by placing the unit in the path of the exhaled breath. This can be achieved by having the subject breath directly into a unit, e.g., through a mouthpiece, or by associating the unit(s) to a ventilation unit to which the subject is connected, or by removing a sample from the subject's lungs. Notwithstanding the means, the exposure may be a single exposure, a plurality of exposures, a continuous exposure over a period of time, or timed exposures, wherein, e.g., the units are exposed at certain time points for a predetermined period.

The duration of each exposure session, e.g. from two different time points, can be the same or different and can vary between several minutes to several hours or more.

Following exposure of the adsorbing regions to a breath sample, the various volatiles become adsorbed on the regions' surface and trapped/associated until identification. Upon need (e.g. for the purpose of identification) the adsorbed volatiles can be desorbed or dissociated by various means, including, without being limited thereto, thermally, under a flow of an inert gas, under vacuum etc.

The identification of the desorbed volatiles can be achieved by any analytical instrument that enables chemical or spectroscopic identification of the desorbed compounds. This may include, without being limited thereto, gas chromatography (GC), GC-lined mass spectrometry (GC-MS), proton transfer reaction mass spectrometry (PTR-MS), electronic nose device, quartz crystal microbalance (QCM), infra-red spectroscopy (IR), ultraviolet spectroscopy (UV) and others.

The identification steps/means can be designed to identify a single volatile compound, two compounds or a plurality of such volatile compounds. The identification can be a chemical identification (e.g. chemical formula) and/or the identification of the peak profile.

In some examples, the analytical instrument comprises MS. In such exemplary cases, a resulting MS chromatogram comprise all separated compounds as chromatographic peaks, arranged by their retention times. Each peak consists of a continuous line connecting several points, wherein each point is the sum of abundances of fragment ion generated from the fragmentation of the material molecules. The peak area is calculated by performing an integral derivative of the abundance of ions according to the time (d_{abundance}/dt) from the starting point of the peak to its end, as derived from Eq. 1:

$${\int }_{PS - T}^{PE - T} \frac{da}{dt} = peak area . \qquad ( Eq . 1 )$$

wherein in Eq. 1, PS-T is the peak start time, PE-T is the peak end time, da is the derivative of the ion's abundance, and dt is the derivative of the retention time.

The identified volatiles, namely, the VOC are then used as input data to be used for the identification of a disease marker profile. Thus, in the context of the present disclosure, when referring to “input data” obtained from a breath sample, it is to be understood to encompass data defining the one or more VOCs identified from one or more breath samples of a specific subject at a specific evaluation session (the session comprising analysis of one or more breath samples).

The input data is then used for querying a database to identify a signatory viral marker profile for the specific virus causing a viral infection.

In the context of the present disclosure, when referring to a “signatory viral marker profile” or in brief “marker profile” it is to be understood to encompass the one or more VOCs that have been a priori clustered together as representing a fingerprint for a virus causing a viral infection that involves replication within a host cell.

For each viral infection, the marker profile can be a priori determined by an in vitro assay where a host cell in a culture media is inoculated with the virus and at a replicating stage, VOCs are identified, both qualitatively and quantitatively. The identification of the VOCs are then compared to a control (“control group”) comprising the identity of background VOCs released from the same host cell and/or medium, without inoculation with the virus (i.e. non-infected cells). The VOCs that are common to those released in the virus infected culture and those from the control group are subtracted from the list of identified VOCs and the remaining VOCs are then considered virus specific and constitute the viral signatory marker profile for the specifically examined virus.

Thus, in accordance with some examples, the in vitro assay comprises inoculating at least one defined host cell in a culture media with a defined virus and determining presence or absence of VOCs released to the surrounding of said culture media during replication of said defined virus strain.

In some further examples, the in vitro assay comprises a plurality of tests, each test comprising inoculating a differently defined host cell in a culture media with a same defined virus strain and determining presence or absence of VOCs released to the surrounding of culture media during replication of said defined virus strain that are common to said plurality of tests.

In some examples, the in vitro method comprises collecting VOCs released from infected host cells during said viral replication. Common VOCs are then used to define the marker profile for the virus strain.

In some further examples, the in vitro method comprises deducing from collected VOCs, the background VOCs, said background VOCs are VOCs released to surrounding of a same culture media in an in vitro assay where said host cells are cultured in said culture media without inoculation with a virus.

The viral signatory marker profile will include VOCs that are not identified from the control group.

The assay can be performed using any culture media suitable for growing the selected host cell. Yet, without being limited thereto, the culture media can be anyone selected from blood agar, Macconkey agar, chocolate agar, Muller hinton, agar, Muller hinton blood agar, mannitol salt agar, Streptococcus selective agar, Sabouraud agar and New York City agar.

In some examples, the assay comprises providing the specified virus in a suitable carrier, be it an appropriate carrier solution, such as culture media or as part of a tissue or lavage material removed from a subject's body, a virus seeds solution, onto to the growth media (liquid agar, agar plate) that is placed in a volume (at times referred to by the term “headspace”) and enabling the virus to grow/replication for a period of from several hours to several days, e.g. for at least 3 hours, at times at least 4 hours, at times at least 7 hours, at times at least 12 hours, at times at least 18 hours, at times for at least one day, for at least 2 days, for at least 3 days, for at least 4 days, and even up to 7 days.

For the identification of the VOCs, the headspace would typically be equipped with a thermal desorption tubes (TDT) such as those described in WO2021/028928 at certain predetermined locations over the growth media. The VOCs that are exhaled by the media of the replicating virus and are then captured and adsorbed by the TDT.

At predetermined frequencies from the beginning of the viral replication the TDT are analyzed for their contents in an analytical unit including optical, electro or optoelectronic device as listed above.

For control group, the growth media itself (with the host cells, however without any virus) is sampled and the VOCs in a respective “control” headspace is analyzed, under the same conditions/parameters of measuring the virus containing media.

For using the above data for elucidating the presence of at least one virus in a patient's' breath further control studies are required. These are identifying the patient's own background, the virus presence in the patient's body prior to sampling his breath. This can be done by taking a tissue of lavage from the patient's body.

When using a headspace of a type described below, measures would need to be taken to exclude from the analysis compounds that are a result of replication of air borne viruses, such as those that can exist in hospital Emergency Room (ER) and intensive care unit (ICU), etc.

In some more specific examples, the association between the specific virus, the specific host cell, and the marker profile can be established according to cell culture protocols known in the art.

Performing the above exemplary protocol for a plurality of clusters of specific virus and specific host cell to obtain the specifically associated marker profile provides the basis for the herein disclosed database. Such database can be periodically updated with any new cluster of virus/host/maker profile as further described below.

Interestingly, it has been found that for a specific virus (e.g. strain), irrespective of the type of host cell, there is a specific marker profile that is associated with the defined virus. Thus, as also exemplified herein, it is possible to identify VOCs that are unique to a virus and will be identified from different host cells infected thereby.

It is further noted, that for a specific virus, it is possible to have more than one viral marker profile, a first marker profile identifying the virus per se (irrespective of the type of host cell), and a second marker profile identifying the virus in associated with a specific host cell, i.e. a set of VOCs that is identified in connection with the specific cell and can be different or partially overlap with another set of VOCs identified in connection with another host cell infected by the same virus.

Thus, in one example, the viral marker profile associated between one or more VOCs and a virus.

In some examples, the viral marker profile associates between one or more VOCs and a group of viruses from a same family.

In some other examples, the marker profile associates between one or more VOCs and a specifically defined virus strain.

In one other example, the viral marker profile associates between one or more VOCs and a virus, preferably virus strain, and a group of host cells infected thereby.

In some further examples, the viral marker profile associates between one or more VOCs, the virus strain and a specific host cell infected by the virus strain.

One particular example of the present disclosure is based on the finding by the inventors that for a group of host cells as well as for a specific host cell, infected with a specific virus, there are unique and distinguishing collection of VOCs, one including VOCs that are common to the group of host cells and one that is unique to the specific host cells.

In some examples, the VOCs are also each characterized by a unique concentration (peak) associated with the virus, and specifically with the state of infection by the virus. It should be understood that VOCs appear as replication is progressing and may then disappear (i.e. when all cells were infected and lysed). There are VOCs whose peak area (correlated amount) increases with progression of infection, the correlated amount then may stabilizes or decreases. There are VOCs that appear and disappear. Hence, detected VOCs appear and are detected depending on the type and nature of the compound(s) that are/is involved and the state/level of the infection.

Further, it has been found by the inventors that a specific virus, infecting different host cells, will provide for each a distinctly different profile of VOCs. In other words, different host cells will present different marker profiles, even if infected by the same specific virus.

Thus, while a specific virus can have a marker profile that is unique to a group of host cells infected thereby, which can be referred to herein as a group level of identification, there is also a host specific level of identification where a marker profile is a priori constructed from VOCs that are unique to a defined virus strain and the host cell being infected thereby.

Accordingly, by querying the database with the specified VOCs (identified from one or more breath or liquid samples), it is possible to essentially unambiguously identify not only the type of infecting virus but also the type of host cell(s) infected thereby.

The database query provides output data specifying the virus and optionally (although preferably) the host cell(s) associated in the database with the virus, i.e. the type of viral infection. The collection of VOCs associated in the database with a virus is referred to as a virus cluster or a viral infection cluster. The database comprises a plurality of such clusters, each cluster defining the specific and unique marker profile for a define virus, and at times, also for defined host cell.

In some examples, the output data also provides at least one treatment protocol against said viral infection and optionally statistical data regarding chances of success of said treatment.

The output data can be compared to a previously determined data, the latter can constitute a reference. In some examples, a statistically significant change in the VOCs for a marker profile as compared to a reference is indicative of an infection's state, as discussed below.

In the context of the present disclosure, when referring to an “infection state” it is to be understood as referring to existence per se of a viral infection, and when compared to a previous determination (i.e. a reference), also to any one of onset of a viral infection, progression in the viral infection, regression of the viral infection, steady state of the viral infection etc.

In some examples, the method steps are performed to establish if the subject has been infected with a virus per se i.e. the mere existence of a viral infection and type of viral infection.

In some other examples, the method steps are performed in order to establish the infection's state, this typically being in comparison with a reference and/or a reference marker profile. In such cases, the determined marker profile is compared to a reference marker; the reference marker can be obtained from of the VOCs identified at an earlier time point for the said subject or based on analysis of breath samples from a statistically significant number of healthy subjects.

A statistically significant change from the reference marker can be indicative of the change in the state of the viral infection.

In the context of the present disclosure, when referring to “an earlier time point” it is to be understood as any time before executing the method steps for the purpose of evaluating the subject's state, the time of executing the method being referred to as the “determination time point” or “evaluation time point”.

In some examples, the evaluation time point is during treatment of the subject against the viral infection and the earlier time point is before initiation of treatment.

In some other examples, the evaluation time point is during treatment of the subject against the viral infection and the earlier time point is at an earlier time point during the treatment.

In some other examples, the evaluation time point is after termination or completion of the anti-viral treatment and the earlier time point is during treatment.

In yet some other examples, the evaluation time point is after termination or completion of the anti-viral treatment and the earlier time point is before initiation of treatment.

In some examples, the comparison of profile markers determined for a subject at two different time points allow for the monitoring the viral infection state. Thus, in accordance with some examples, the present disclosure provides a method for monitoring disease progression and/or monitoring treatment against a viral infection in a subject, the method comprises:
a. obtaining from a breath sample of said subject input data specifying one or more VOCs said sample being obtained at a determination time point;
b. querying a database using said input data to identify a marker profile associated with the at least one of said specified one or more VOCs;
c. contingent upon the identification of a marker profile associated with said specified one or more VOCs, providing output data with respect to the defined virus and optionally the defined host cell; and
d. comparing said output data with prior data corresponding to a marker profile obtained for said subject at a time preceding the determination time point; wherein a change in said marker profile being indicative of a viral infection state.

The method disclosed herein can also be employed for determining a treatment protocol for a subject being suspected of having a viral infection. In accordance with this example, the method comprises:
a. determining for the subject a viral marker profile, the determination comprises:
   i. from said breath sample obtaining input data specifying one or more VOCs;
   ii. querying a database using said input data to identify a viral marker profile, the marker profile being associated in said database with the one or more VOCs; and
   iii. contingent upon identification of the viral marker profile, providing output data with respect to the defined virus and optionally the defined host cell; said output data comprises determination of type of viral infection in said subject; and
b. determining or selecting a treatment protocol for the identified viral infection.

In yet some other examples, the method disclosed herein can be utilized for treating a subject having a viral infection, the treatment method comprising providing the subject with an anti-viral treatment, wherein the anti-viral treatment is determined by:
a. obtaining input data specifying one or more VOCs identified from a breath sample of said subject;
b. querying a database using said input data for a viral marker profile, the viral marker profile being associated with the one or more specified VOCs; and
c. contingent upon identification of the viral marker profile, providing output data with respect to the defined virus and optionally the defined host cell; and
d. determining based on said output data an anti-viral treatment suitable for said viral infection in said subject.

The basis of the present technology resides in the establishment of a database providing the unique association between a profile marker, a specific virus and a specific host cell. The database disclosed herein can be employed for any determination relating to a viral infection, this including without being limited thereto determination of actual existence of an infection, determining treatment protocol, monitoring treatment of disease, monitoring progression of a disease, monitoring regression of a disease etc.

The database disclosed herein comprises a structured collection of records including a bank of viral marker profiles associated with a bank of defined viruses, wherein said marker profile comprises specified VOCs that are released from a defined host cell, during viral replication with a defined virus.

As described hereinabove, the database is constructed by associating a unique marker profile with specific host cell(s) and specific virus.

The database may also comprise information regarding types of treatment to be used against a specific viral infection, statistical information regarding the success rate of a particular treatment, alternative treatments, etc.

In some examples, as also described above, each unique marker profile can be pre-associated with a viral infection (virus/host cell) in an in vitro assay comprising identification of the one or more VOC specifying the marker profile from debris of a defined host cell infected with a defined virus causing the viral infection.

In some examples, the database comprises a bank of treatment protocols, each treatment protocol being associated with a cluster comprising a marker profile, a defined host cell and a defined virus, and a priori determined to be the treatment with the highest prospects of defeating the specific viral infection with which it is associated.

In some examples, the database comprises an identification cluster for beta coronavirus, such as CoronaOC43 virus infecting in alveolar epithelium cells, as exemplified herein with A549 cells (human cells).

In some examples, the database comprises an identification cluster for beta coronavirus, such as CoronaOC43 virus infecting colorectal adenocarcinoma cells HCT8.

In some examples, the database comprises an identification cluster for coronavirus, such as a beta coronavirus, and particularly CoronaOC43 virus infecting fibroblast cells, e.g. in the lungs, as exemplified with MRC5 cells (normal fibroblast cells from lung tissue).

In some examples, the database comprises the association of a virus with more than one host cell. For example, as shown herein, beta coronavirus, such as CoronaOC43 virus infecting each of the HCT8 (colorectal adenocarcinopma cells), A549 (human cells), MRC5 cells, where each virus infecting a different type of cell provides its unique marker profile. It was further found that the association of a virus with more than one host cell has exhibited two commonly specified VOCs (1) C₉H₁₃NO having a retention time of 5.27 minutes and C₈H₄N₂ having a retention time of 18.37 minutes, common to these three exemplified cells HCT8, A549, MRC5 cells when infected with an identical virus. Therefore, two compounds originate from the virus and are not specific to a host cell. This finding supports the understanding that the debris of cells in which a virus is replicated release volatiles that are specific to a virus, irrespective of the volatiles released from the host cell per se.

Table 1A provides marker profiles (i.e. list of VOCs and SVOCs) for coronavirus OC43 infected cells with different host cells. The listed compounds are those identified with a probability of >80%, as indicated by the level of match.

In the context of the present disclosure, the marker profile comprises one or combination of the compounds within the list of compounds presented in Table 1A, as long as the one or combination of compounds is sufficiently distinctive (e.g. when considering also the relative amount of the compound, i.e. mass to charge ratio, m/z) to specifically identify the virus and host cells associated therewith.

TABLE 1A
Clusters for Corona OC43

| | | | Cell Type | | |
|---|---|---|---|---|---|
| Compound | CAS# | Match | A549 | HCT8 | MRC5 |
| alpha-Methylstyrene | 98-83-9 | 90.70 | | | 16.012 |
| 1,2-Benzenedicarbonitrile | 91-15-6 | 81.53 | | 18.356 | |
| 1-Chloroundecane | 2473-02-3 | 81.06 | | | 19.942 |
| 1H-Cyclopropa[a]naphthalene, | 20071-49-2 | 87.67 | | 22.659 | |
| decahydro-1,1,3a-trimethyl-7- | | | | | |
| methylene-, [1aS- | | | | | |
| (1aα,3aα,7aβ,7bα)]- | | | | | |
| 1-Hexanol, 2-ethyl- | 104-76-7 | 92.58 | | | 13.595 |
| 1-Phenanthrenecarboxylic acid, | 3582-27-2 | 80.88 | 31.786 | | |
| 7-ethyl- | | | | | |
| 1,2,3,4,4a,4b,5,6,7,9,10,10a- | | | | | |
| dodecahydro-1,4a,7-trimethyl-, | | | | | |
| methyl ester, [1R- | | | | | |
| (1α,4aβ,4bα,7β,7aα)]- | | | | | |
| 1-Propanol, dl-2-benzylamino- | 6940-81-4 | 82.73 | | 30.252 | |
| 2-Ethylhexyl acrylate | 103-11-7 | 96.74 | | 19.195 | |
| 2-Ethyl-oxetane | 1000386-40-2 | 92.08 | | | 1.818 |
| 2-Methyltetracosane | 1560-78-7 | 90.89 | | | 32.579 |
| 3-Methylheptyl acetate | 72218-58-7 | 83.39 | | 17.246 | |
| Acetic acid, 2-ethylhexyl ester | 103-09-3 | 87.04 | | 17.233 | |
| Acetophenone | 98-86-2 | 97.48 | | | 14.963 |
| Benzene, 1,3,5-trichloro- | 108-70-3 | 86.91 | 18.107 | | |
| propyl-Benzene, | 103-65-1 | 89.25 | | | 10.599 |
| Benzeneacetaldehyde | 122-78-1 | 90.77 | | 10.602 | |
| Benzenemethanol, α,α4- | 1197-01-9 | 91.03 | | 18.528 | |
| trimethyl- | | | | | |
| Benzenemethanol, α,α-dimethyl- | 617-94-7 | 90.10 | | | 15.595 |
| Benzyl alcohol | 100-51-6 | 89.76 | 14.470 | | |
| Bicyclo[4.1.0]hept-2-ene, 3,7,7- | 4497-92-1 | 96.12 | | 15.170 | |
| trimethyl-, (1S-cis)- | | | | | |
| Bromochloronitromethane | 135531-25-8 | 86.04 | | | 5.234 |
| Butane, 2,3-dimethyl- | 79-29-8 | 81.52 | | | 1.685 |
| Carvone | 99-49-0 | 81.59 | | 19.778 | |
| Cyclopropane, 1,2-dimethyl-, | 2402-06-4 | 87.65 | | 4.423 | |
| trans- | | | | | |
| Decane, 2,3,5,8-tetramethyl- | 192823-15-7 | 91.75 | | | 23.246 |
| Decane, 2-methyl- | 6975-98-0 | 88.35 | | | 14.288 |
| Decane, 3-bromo- | 30571-71-2 | 82.73 | | 19.429 | |
| Dimethyl sulfide | 75-18-3 | 82.62 | 1.595 | | |
| Dodecane | 112-40-3 | 90.45 | | | 18.373 |
| Ethyl Acetate | 141-78-6 | 96.34 | | | 1.967 |
| Furfurylmethylamphetamine | 13445-60-8 | 80.72 | | 11.985 | |
| Heptane, 2,2,4,6,6-pentamethyl- | 13475-82-6 | 96.30 | | 11.647 | |
| Heptane, 2,4-dimethyl- | 2213-23-2 | 89.39 | | | 5.432 |
| Heptane, 3,3,5-trimethyl- | 7154-80-5 | 82.85 | | 12.850 | |
| Heptane, 3-methylene- | 1632-16-2 | 91.38 | | 4.423 | |
| Heptane, 4-ethyl- | 2216-32-2 | 82.66 | | | 12.511 |
| Hexane, 2,2,5-trimethyl- | 3522-94-9 | 80.09 | | 13.822 | |
| Isopropyl palmitate | 142-91-6 | 98.10 | | 29.319 | |
| Longifolene | 475-20-7 | 90.33 | 22.410 | | |
| Methane, bromodichloro- | 75-27-4 | 96.67 | | | 3.014 |
| Methane, dibromochloro- | 124-48-1 | 84.12 | | | 5.250 |
| n-Hexane | 110-54-3 | 94.69 | 1.827 | | |
| Nonane, 2,6-dimethyl- | 17302-28-2 | 90.27 | | | 12.816 |
| Nonane, 2-methyl- | 871-83-0 | 90.64 | | | 10.817 |
| o-Cymene | 527-84-4 | 84.65 | | | 13.133 |
| Oxalic acid, isobutyl pentyl ester | 1000309-37-0 | 87.06 | 8.427 | | |
| Pentane, 2-methyl- | 107-83-5 | 81.63 | | | 1.685 |
| Pentane, 3-methyl- | 96-14-0 | 95.49 | | | 1.747 |
| p-Xylene | 106-42-3 | 97.21 | | | 7.497 |
| Squalene | 111-02-4 | 95.26 | | 30.796 | |
| Sulfurous acid, 2-ethylhexyl | 1000309-19-2 | 80.33 | | | 15.404 |
| nonyl ester | | | | | |
| Tricyclo[5.4.0.0(2,8)]undec-9- | 5989-02-8 | 87.12 | | 21.860 | |
| ene, 2,6,6,9-tetramethyl-, | | | | | |
| (1R,2S,7R,8R)- | | | | | |
| Tridecane, 3-methyl- | 6418-41-3 | 92.36 | | 21.616 | |

In some examples, the database comprises an identification cluster for alpha coronavirus, such as Corona229E virus infecting in alveolar epithelium cells.

Table 1B provides marker profiles (i.e. list of VOCs and SVOCs) for coronavirus 229E infected cells with different host cells. Also here, the listed compounds are those identified with a probability of >80%, as indicated by the level of match.

In the context of the present disclosure, the marker profile comprises one or combination of the compounds within the list of compounds presented in Table 1B, as long as the one or combination of compounds is sufficiently distinctive (e.g. when considering also the relative amount of the compound) to specifically identify the virus and host cells associated therewith.

TABLE 1B
Clusters for Corona 229E

| | | | Cell Type | |
|---|---|---|---|---|
| Compound (probability >80%) | CAS# | Match | A549 | HCT8 |
| (1S,4aS,4bS,7S,8aS,10aS)-7-Isopropyl- | 2221-95-6 | 91.25 | 28.661 | |
| 1,4a-dimethyltetradecahydrophenanthrene | | | | |
| 1-Chloroundecane | 2473-03-2 | 94.35 | 19.934 | |
| 1-Methoxy-2-propyl acetate | 108-65-6 | 87.45 | 7.855 | |
| 1-Phenanthrenecarboxylic acid, | 33892-15-8 | 81.26 | 32.045 | |
| 1,2,3,4,4a,5,6,7,8,9,10,10a-dodecahydro- | | | | |
| 1,4a-dimethyl-7-(1-methylethyl)-, methyl | | | | |
| ester, [1R-(1α,4aβ,7β,10aα)]- | | | | |
| 1-Phenanthrenecarboxylic acid, 7-ethyl- | 3582-27-2 | 83.25 | 31.760 | |
| 1,2,3,4,4a,4b,5,6,7,9,10,10a-dodecahydro- | | | | |
| 1,4a,7-trimethyl-, methyl ester, [1R- | | | | |
| (1α,4aβ,4bα.,7β,7aα)]- | | | | |
| 2-Methylhexacosane | 1561-02-0 | 88.35 | 33.436 | |
| 4-Bromoheptane | 998-93-6 | 82.61 | 22.750 | |
| 7-Methyl-Z-tetradecen-1-ol acetate | 1000130-99-6 | 80.02 | 32.391 | |
| Benzene, 1,1′-[1,2-ethanediylbis(oxy)]bis- | 104-66-5 | 84.53 | | 27.429 |
| Benzene, 1,3,5-trichloro- | 108-70-3 | 83.43 | 18.112 | |
| Benzene, 1,4-dichloro- | 106-46-7 | 95.69 | 12.598 | |
| Benzoic acid, ethyl ester | 93-89-0 | 92.91 | 17.999 | |
| Benzyl alcohol | 100-51-6 | 80.84 | 14.528 | |
| Cyclotetradecane | 295-17-0 | 87.52 | 22.840 | |
| Di-epi-α-cedrene | 50894-66-1 | 81.01 | | 21.876 |
| Diphenylamine | 122-39-4 | 82.94 | 25.600 | |
| Dodecane | 112-40-3 | 96.41 | 18.382 | |
| Kaur-16-en-18-al, (4α)- | 14046-84-5 | 82.03 | 32.202 | |
| Longifolene | 475-20-7 | 94.30 | 22.417 | |
| Methacrolein | 78-85-3 | 83.50 | 1.781 | |
| Methane, isocyanato- | 624-83-9 | 90.93 | 1.775 | |
| Pentane, 3-methyl- | 96-14-0 | 82.10 | | 1.756 |
| Podocarpan-15-oic acid, 13-isopropyl-, | 32208-29-0 | 80.27 | 31.966 | |
| methyl ester | | | | |
| Tetradecane | 629-59-4 | 97.16 | 22.066 | |
| Tridecane | 629-50-5 | 95.78 | 20.438 | |
| Tridecane, 2-methyl- | 1560-96-9 | 92.96 | 21.497 | |
| Tridecane, 3-methyl- | 6418-41-3 | 93.37 | 21.605 | |
| Undecane, 3,8-dimethyl- | 17301-30-3 | 82.86 | 26.315 | |
| Undecane, 4,4-dimethyl- | 17312-68-4 | 83.88 | 21.314 | |

In yet some other examples, the database comprises an identification cluster for influenza virus, such as influenza A (H1N1) strain infecting in alveolar epithelium cells.

Table 1C provides marker profiles (i.e. list of VOCs) for influenza A H1N1 infected A549 cells. Also here, the listed compounds are those identified with a probability of >60%, as indicated by the level of match.

In the context of the present disclosure, the marker profile comprises one or combination of the compounds within the list of compounds presented in Table 1C, as long as the one or combination of compounds is sufficiently distinctive (e.g. when considering also the relative amount of the compound) to specifically identify the virus and host cells associated therewith.

TABLE 1C
Cluster for Influenza A H1N1

| | | | Cell Type |
|---|---|---|---|
| Compound (probability >60%) | CAS# | Match | A549 |
| [1,1′:3′,1″-Terphenyl]-2′-ol | 2432-11-3 | 83.53 | 31.658 |
| 1,5-Dioxaspiro[5.5]undecan-9-one, 3,3- | 69225-59-8 | 70.80 | 19.384 |
| dimethyl- | | | |
| 1-Butanol, 3-methyl-, benzoate | 94-46-2 | 79.23 | 27.077 |
| 1-Dodecanamine, N,N-dimethyl- | 112-18-5 | 88.33 | 23.739 |
| 1-Phenanthrenecarboxylic acid, | 2651-34-5 | 60.58 | 31.031 |
| 1,2,3,4,4a,9,10,10a-octahydro-1,4a-dimethyl-, | | | |
| methyl ester, [1S-(1α,4aα,10aβ)]- | | | |
| 2,3-Dimethylhydroquinone, bis(trimethylsilyl) | 140901-63-9 | 76.41 | 11.309 |
| ether | | | |
| 2-Fluorobenzoic acid. heptadecyl ester | 1000282-96-6 | 69.66 | 20.548 |
| 2-Methyl-β-alanine, N-benzyl-N-methyl-, | 1000452-75-6 | 60.59 | 30.239 |
| methyl ester | | | |
| Benzene, 1,2,3,5-tetramethyl- | 527-53-7 | 61.93 | 17.262 |
| Benzeneacetaldehyde | 122-78-1 | 90.40 | 10.584 |
| Benzyl butyl phthalate | 85-68-7 | 72.80 | 32.438 |
| Butyric acid, 2-phenyl-, 4-nitrophenyl ester | 1000406-87-5 | 61.68 | 28.849 |
| Dodecane, 1-chloro- | 112-52-7 | 98.29 | 23.175 |
| Heneicosane | 629-94-7 | 97.48 | 32.534 |
| L-Proline, ethyl ester | 5817-26-5 | 62.22 | 6.173 |
| Propanedioic acid, amino-, diethyl ester | 6829-40-9 | 60.47 | 6.844 |
| Tetradecane, 1-chloro- | 2425-54-9 | 83.90 | 23.187 |
| Tetradecanoic acid | 544-63-8 | 70.54 | 26.748 |

Similarly, Table 1D provides marker profiles (i.e. list of VOCs) for SARS Corona virus-2 (causing Covid-19) WT (wild type virus infected Vero cells (epithelial kidney cells) grown on a medium of MEM-Eagle Earle Salt Base (red medium)+2% FCS/FBS (fetal bovine serum). Also here, the listed compounds are those identified with a probability of >60%, as indicated by the level of match.

In the context of the present disclosure, the marker profile comprises one or combination of the compounds within the list of compounds presented in Table 1D, as long as the one or combination of compounds is sufficiently distinctive (e.g. when considering also the relative amount of the compound) to specifically identify the virus and host cells associated therewith.

TABLE 1D
Cluster for Corona virus (SARS CoV2) -WT

| Compound Name | CAS # | Formula |
|---|---|---|
| 2-ethyltetrahydrofurna | 1000431-64-0 | C₆H₁₂O |
| Isovaleric acid, 3-methtylbutyl-2-ester | 1000360-64-9 | C₁₀H₂₀O₂ |
| (+/−)-2-phenylpropanoic acid, iso-propyl | 1000453-23-1 | C₁₂H₁₆O₂ |
| ester | | |
| 1,1′-di-tert-butyl ferrocene | 1000148-69-0 | C₁₈H₂₆Fe |
| Cyclohexane, 1α,2α,4α,5α-tetraethyl | 61142-24-3 | C₁₄H₂₈ |
| 3,5-diphenyl-1H-pyridazin-4-one | 961-01-3 | C₁₆H₁₂N₂O |
| 2H-benzofuran[3,2-E]1,4-diazepin-2-one, | 57826-69-4 | C₁₇H₁₂N₂O₂ |
| 1,3-dihydro-5-phenyl- | | |
| 2,5-hexanedione | 110-13-4 | C₆H₁₀O₂ |
| 4-cycvlopentene-1,3-dione, 2,4-diphenyl | 80213-58-7 | C₁₇H₁₂O₂ |
| 2,5-cyclohexadiene-1,4-dione,2,5-diphenyl | 844-51-9 | C₁₈H1₁₂O₂ |
| 4-anilinoquinazoline | 34923-95-0 | C₁₄H₁₁N₃ |
| Methanone, phenyl(5-phenyl-1H-pyrazol- | 21111-32-0 | C₁₆H₁₂N₂O |
| 3-yl) | | |
| 2,2′,3,3′,5,5′,6,6′-octafluorobiphenyl | 3883-86-1 | C₁₂H₂F₈ |
| 4-propyl-Phenol, | 645-56-7 | C₉H₁₂O |

Similarly, Table 1E provides marker profiles (i.e. list of VOCs) for Corona virus (Covid-19) UK type infected Vero cells (epithelial kidney cells) grown on a medium of MEM-Eagle Earle Salt Base (red medium)+2% FCS/FBS (fetal bovine serum). Also here, the listed compounds are those identified with a probability of >60%, as indicated by the level of match.

In the context of the present disclosure, the marker profile comprises one or combination of the compounds within the list of compounds presented in Table 1E, as long as the one or combination of compounds is sufficiently distinctive (e.g. when considering also the relative amount of the compound) to specifically identify the virus and host cells associated therewith.

TABLE 1E
Cluster for Corona virus (SARS CoV2) -UK type

| Compound name | CAS # | formula |
|---|---|---|
| 2-ethyl-hexanol | 104-76-7 | C₈H₁₈O |
| (S)-(+)-1-(2-pyrrolidinylmethyl)- | 51207-66-0 | C₉H₁₈N₂ |
| pyrrolidine | | |
| Oxalic acid, decyl-1-menthyl ester | 1000314-97-8 | C₂₂H₄₀O₄ |
| Benzamide,4-butyl-N-(2- | 1000451-40-4 | C₂₂H₂₉NO |
| phenylethyl)-N-propyl | | |
| Methylene-cyclopentane | 1528-30-9 | C₆H₁₀ |
| Pyrrolidine-1-acetic acid | 37386-15-5 | C₆H₁₁NO₂ |
| 1,2-dimethyl-3-pentyl-4-propyl- | 62376-17-4 | C₁₆H₃₂ |
| cyclohexane, | | |

The present disclosure also provides a system.

The system is typically computer-based using a database as defined herein above and is also to be understood as a structured collection of records includes, inter alia, a bank of defined viruses associates with a bank of defined host cells, the structured collection of records being stored on a memory unit.

In some examples, the system comprises
a. an input interface for receiving input data specifying one or more VOCs;
b. a management module comprising a database responsive to a query specifying the one or more VOCs for returning a respective marker profile;
c. an output provisioning module adapted to provide as output data any one or combination of a defined virus and optionally a defined host cell; each being associated with a marker profile; wherein said database comprises a structured collection of records including a bank of marker profiles associated with a bank of defined viruses, and with a bank of defined host cells, wherein said marker profile comprises specified VOCs that are released from a defined host cell, during viral replication with a defined virus.

The input interface can be adapted to obtain or receive data with respect to a VOCs. The data with respect to the specified VOCs can be automatically extracted from a pre-stored digital data source, such as a structured file (e.g., a form including content and metadata) or it may be manually input by an operator/user of the system.

The data with respect to the specified compounds may be locally fed to the system, for example, through a keyboard directly connected to the system (“on site”), or the data with respect to the specified compounds may be obtained from a remote location, for example, from a remote computer operatively connected to the system over a communication network, for example, the World Wide Web (WWW). In some examples, the system can obtain further information related to the subject, such as the subject's gender, age, ethical background, virus subtype, chronic diseases, treatment, vaccination.

In some examples, the management module, comprising the database, also comprises a processing utility for receiving and processing data relating to specified VOCs and analyzing the data by running a dedicated software application that performs the analysis and storage of incoming data. The software application may be embodied in a program storage device readable by machine, such as a memory disk.

The processing utility also outputs at least one output data including any one or combination of type of viral infection, type of treatment, statistical data regarding rate of success of treatment, as well as other information of interest.

The processing utility is connected to an input utility and to an output utility. The input utility is typically a user interface unit, such as a keyboard or touch screen to be used for inputting information relating to the specific evaluation session (specified compounds, details of the examined subject etc).

The output utility may be composed of a number (combined or alternative) of digital output units, including computer and/or wearable digital display unit.

### DETAILED DESCRIPTION OF NON-LIMITING EXAMPLES

### In Vitro Assay Module

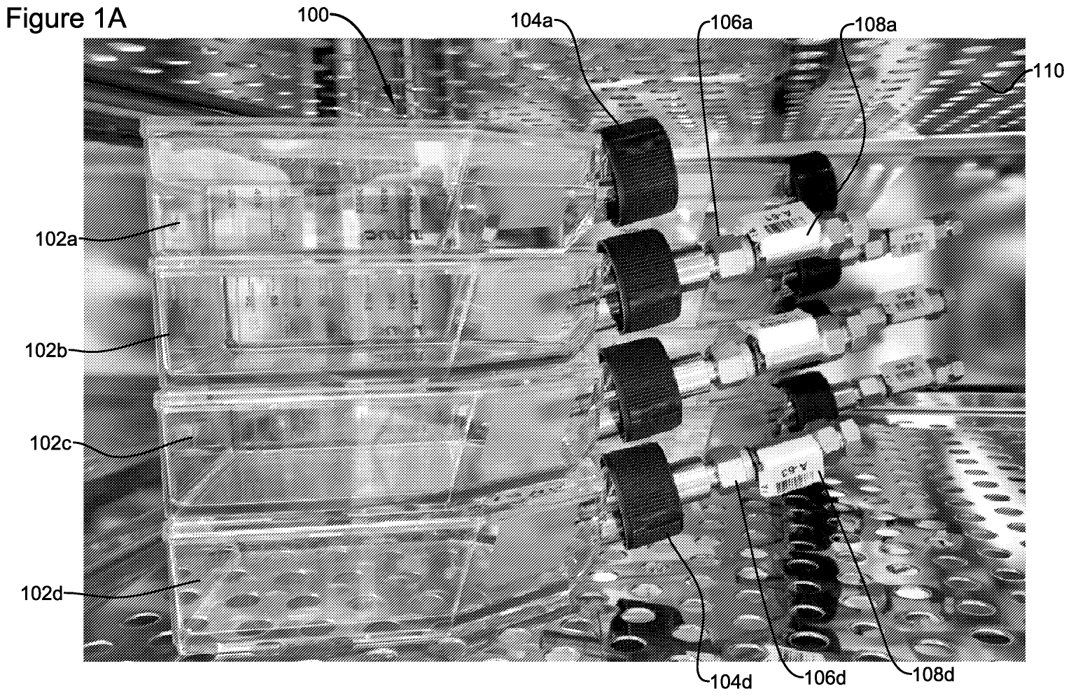

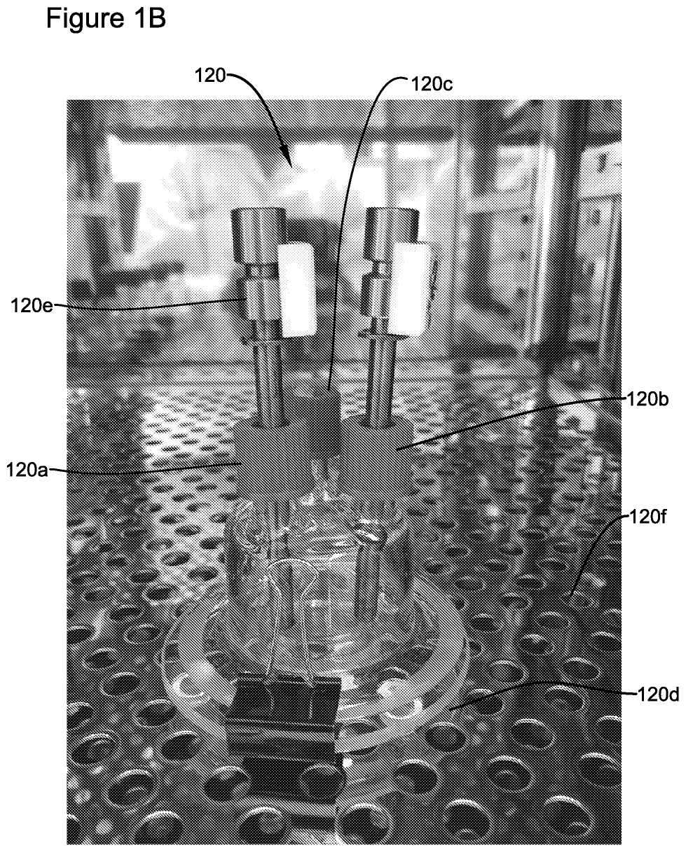

The construction of a database as disclosed herein can utilize a multi-array culture system, such as that shown in FIG. 1A. Specifically, the multi array culture system 100 includes 4 culturing flasks, 102a, 102b, 102c and 102d, one stacked over another, each flask includes a respective cap 104a, 104b, 104c and 104d, a respective connector 106a, 106b, 106c and 106d, and a respective thermal desorption tube (TDT) 108a, 108b, 108c and 108d of a type described in WO2021/028928, such that each flask is sealed on the distal end of each tube.

In operation, prior to sealing each flask 102a, 102b, 102c and 102d, with the respective cap, connector and TDT connected to a collection unit (CU, not shown), each flask is either remained empty (Control #1) or filled with the desired test medium: cell growth medium only (Control #2), cell growth medium with host cells only (Control #3) or cell growth medium with host cells and defined virus during replication thereof (Test). The VOCs from each test flask is collected via the TDT while being maintained in an incubator at a temperature of 33° C.

The analysis comprises identified the VOCs collected from the Control flasks (#1-#3) and subtracting these VOCs those collected in the Test group. The remaining collection of VOCs are then defined as the VOCs specific to the virus the defined host cell.

In an alternative example, the database can be constructed utilizing a multi-array culture system such as that shown in FIG. 1B. Specifically, FIG. 1B shows a multi array culture system 120 includes three caps 120a, 120b, and 120c connected to respective connector and a respective thermal desorption tube (TDT) as described with respect to FIG. 1A, each of the TDT connected to a culture petri dish 120d. The operation of this system can be similar to that described with respect to FIG. 1A.

### EXAMPLES

In the following Examples, for different flask groups were employed:
Control #1: Cell growth medium only
Control #2: Cell growth medium with cells
Control #3: Empty flask
Test: Cell growth medium with cells and replicating virus.

Each example was performed using 30*75 cm² flasks equipped with the Thermal Desorption Tube/Collection Unit (TDT/CU) and conducted according to the conditions specified in Table 2.

### Materials:

Culture Medium: MEM-Eagle Earle Salt Base (red medium)+2% FCS/FBS (fetal bovine serum); or Fetal Bovine Serum (FBS) medium
Host Cells: A549 cells, HCT8 cells, Vero cells, all cells obtained form Sheba Hospital, Israel.
Virus: 229E coronavirus, OC43 Coronavirus, SARS-Cov-2 (WT), all viruses obtained from Sheba Hospital, Israel

The different combination of cells and viruses were assayed according to Tables 2A-2C.

TABLE 2A
In vitro Assay Conditions A549 cells/229E coronavirus

| | No. | |
|---|---|---|
| Group | flasks | Conditions |
| Control | 12 | 2 ml of EMEM Medium (MEM-NEAA, Earle's salts, non-essential amino |
| | | acids Cat No: BI Cat # 01-040-1A) with 250,000-500,000 A549 cells in |
| | | 35 mm petri dish |
| Test | 12 | 229E solution (Virus/cells ration of 0.001-0.002) with 2 ml of EMEM |
| | | Medium (MEM-NEAA, Earle's salts, non-essential amino acids Cat No: BI |
| | | Cat # 01-040-1A) with 250,000-500,000 A549 cells in 35 mm petri dish |

TABLE 2B
In vitro Assay Conditions HCT8 cells/OC43 coronavirus

| | No. | |
|---|---|---|
| Group | flasks | Conditions |
| Control | 12 | 2 ml of EMEM Medium (MEM-NEAA, Earle's salts, non-essential amino |
| | | acids Cat No: BI Cat # 01-040-1A) with 250,000-500,000 HCT8 cells in |
| | | 35 mm petri dish |
| Test | 12 | OC43 solution (Virus/cells ration of 0.001-0.002) with 2 ml of EMEM |
| | | Medium (MEM-NEAA, Earle's salts, non-essential amino acids Cat No: BI |
| | | Cat # 01-040-1A) with 250,000-500,000 HCT8 cells in 35 mm petri dish |

### Samplings:

TABLE 2C
In vitro Assay Conditions Vero cells/SARS-Cov-2

| | No. | |
|---|---|---|
| Group | flasks | Conditions |
| Control#1 | 9 | 12 ml of 2% FBS Medium in 75 cm² flask |
| Control#2 | 9 | 12 ml of 2% FBS Medium with 80,000,000 Vero cells |
| | | in 75 cm² flask |
| Control#3 | 3 | Empty in 75 cm² flask in incubator |
| Test | 9 | 250 μl of SARS-Cov-2 (WT) solution with 12 ml of |
| | | 2% FBS Medium with 80,000,000 Vero cells in in 75 |
| | | cm² flask |

Every specified time point (hours) of incubation, a specified amount of flasks from Control #1, Control #2 and Test groups were removed from the incubator for sampling. The sampling is performed as described in WO 2021//028928, the content of which is incorporated herein by reference.

Briefly, the TDT absorbed metabolites form each flask is released by treating the adsorbing regions to cause desorption or dissociation of the volatiles from the surface, and the volatiles are thereafter analyzed. Desorption or dissociation of the volatiles from the adsorbing regions may be achieved thermally, under a flow of an inert gas, under vacuum or by employing any means that can release the volatiles into the analyzer, all of which is as described in WO 2021//028928, the content of which is incorporated herein by reference.

The analysis of the VOCs was conducted using Thermal Desorption Autosampler which is connected to Quadrupole GC-MS system. equipped with a column selected The separation was done using SGE PN 99054140, 20M×0.18mmID-BPX5×0.18 μm df, with He flow of 0.5 ml/min (Constant flow/pressure).

The retention time and the mass spectra of the VOCs & SVOCs compounds was then used to identify and distinguish between VOCs, using GCMS deconvolution software.

### Results

The identified VOCs for each tested virus and host cells are presented in the following Figures.

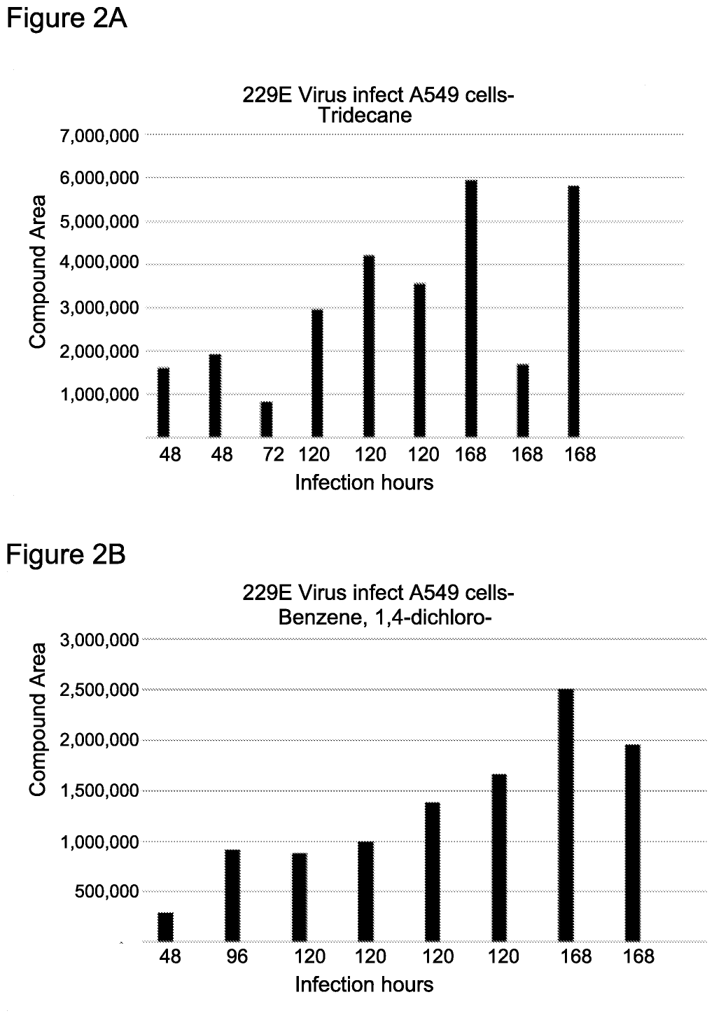

FIGS. 2A-2B identify two VOCs specific to A549 cells infected with replicating 229E virus. FIG. 2A shows the identification of Tridecane compound and FIG. 2B shows the identification of 1,4-dichloro-benzene compound, both already after 48 hours of incubation, and their presence continued also after 168 hours of incubation. A more comprehensive list of compounds is listed in Table 1B above.

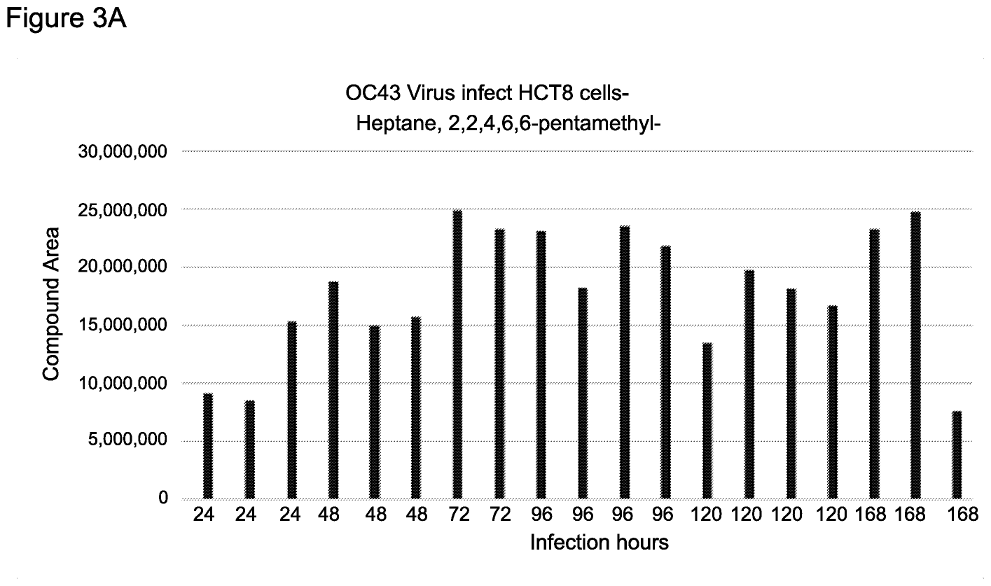

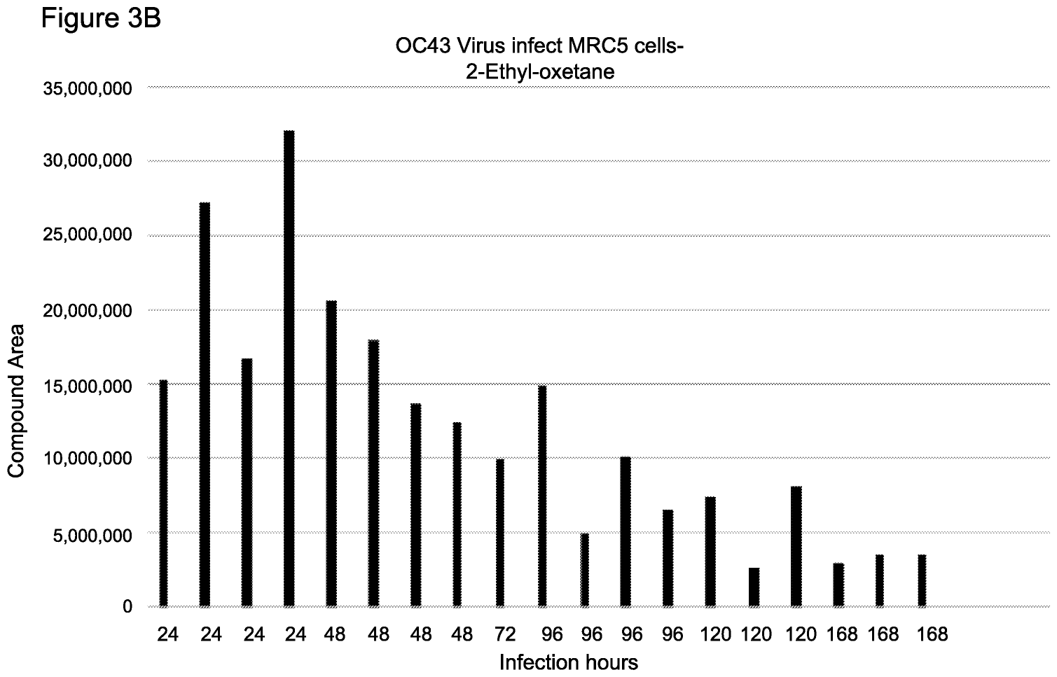

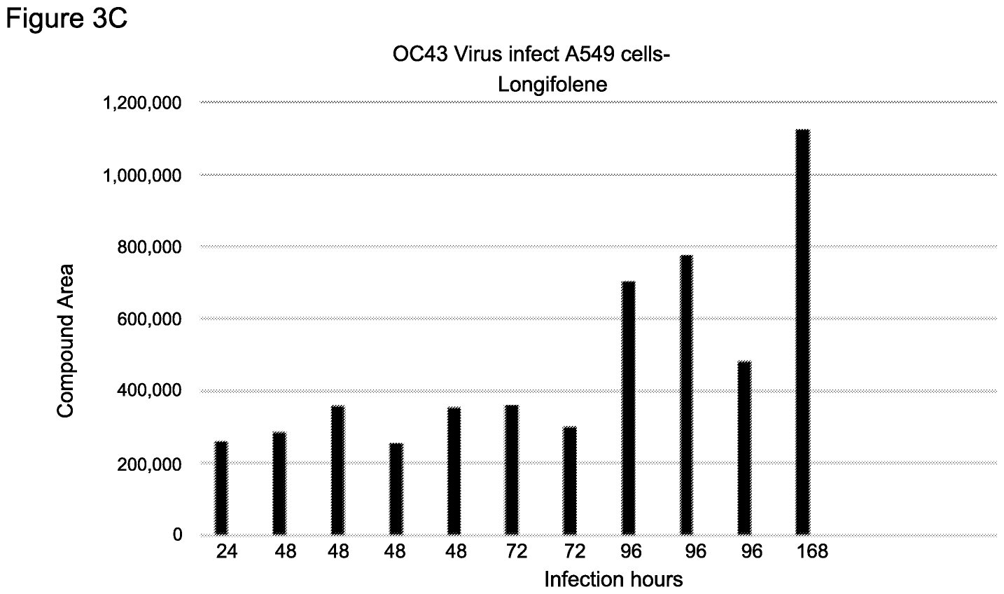

FIGS. 3A-3C identify three VOCs specific to HCT8 cells infected with replicating OC43 virus. FIG. 3A shows the identification of 2,2,4,6,6-pentamethyl-heptane compound; FIG. 3B shows the identification of 2-ethyl-oxetane compound, and FIG. 3C shows the identification of Longifolene (C₁₅H₂₄), all three already after 24 hours of incubation, and their presence continued also after 168 hours of incubation. A more comprehensive list of compounds is listed in table 1A above.

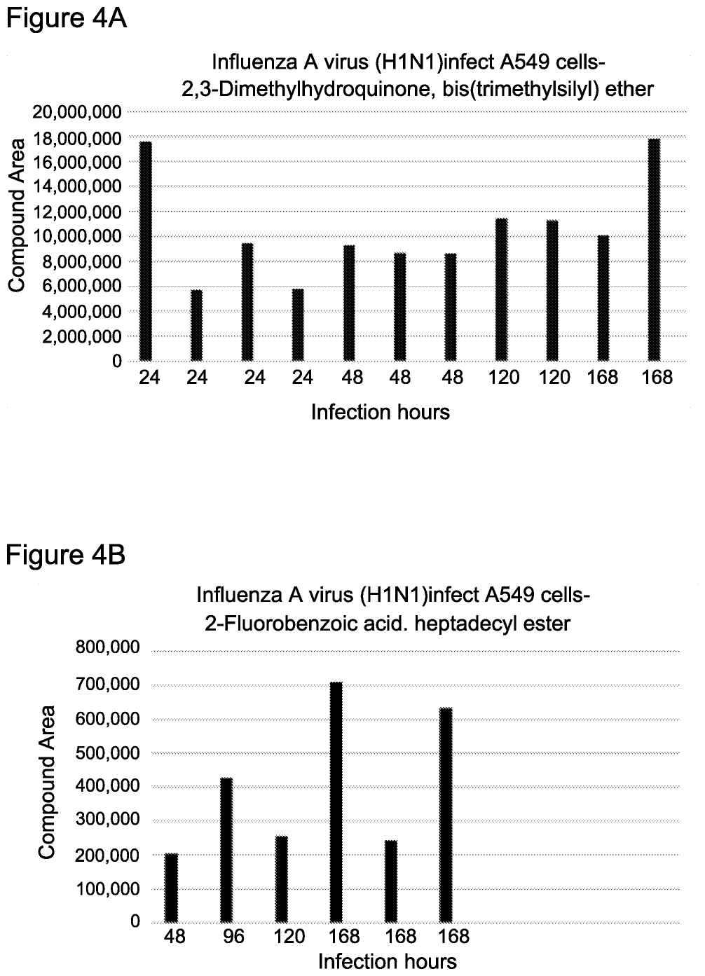

FIGS. 4A-4B identify three VOCs specific to A549 cells infected with replicating HIN1 Influenza A virus. FIG. 4A shows the identification of Heptadecyl ester of 2-fluorobenzoic acid compound while FIG. 4B shows the identification of Bis-(trimethylsilyl) ether of 2,3-dimethyihydroquinone compound, both identified already after 24 hours of incubation, and their presence continued also after 168 hours of incubation. A more comprehensive list of compounds is listed in Table 1C above.

The VOCs of SARS-Cov-2 where identified and these are listed in Tables 1D-1E above.

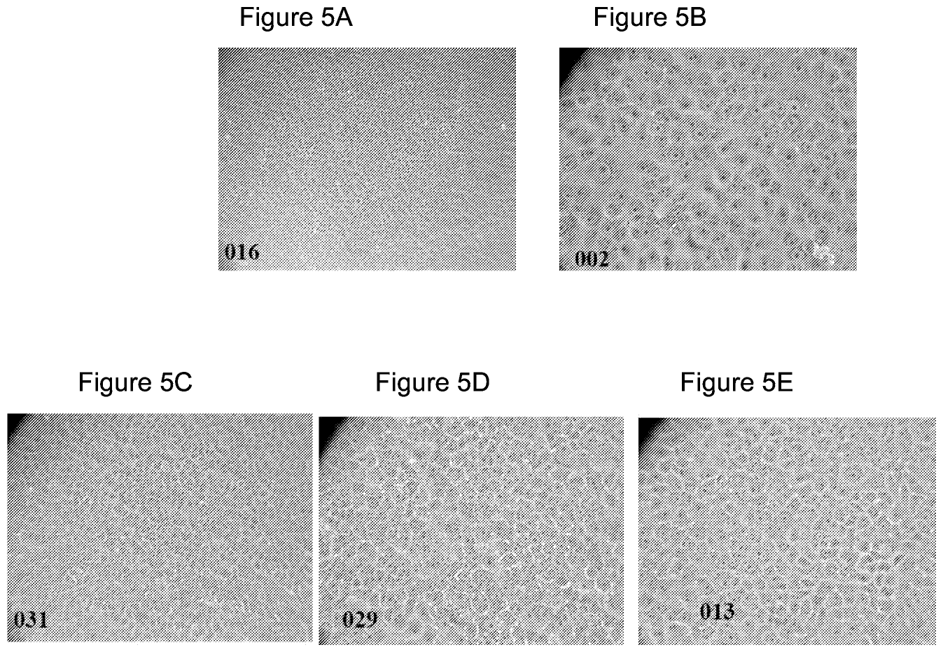

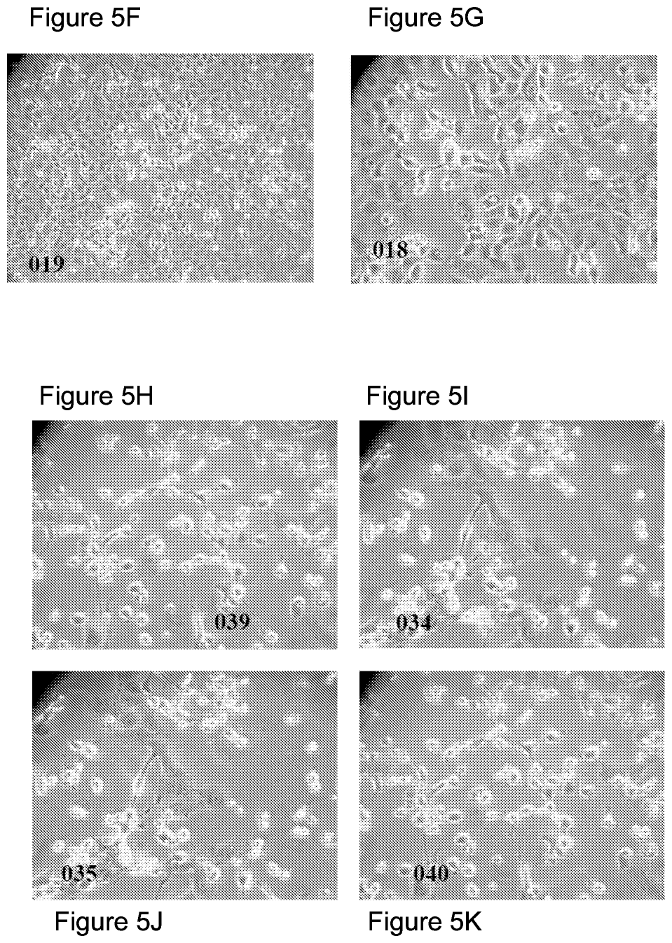

Further, the lytic effect of SARS CoV-2 on Vero cells was imaged, to support the understanding that the VOCs are in fact released only during replication—and host cell lysis. Specifically, images at zero time point (FIGS. 5A, 5B), after 24 hours (FIGS. 5C, 5D, 5E), after 48 hours (FIGS. 5F, 5G) and after 72 hours (FIGS. 5H, 5I, 5J, 5K) of incubation were captured. At each time two, three or four images are given that display different enlargement (zooming in) of the same cells.

The various images clearly illustrate that in time the number of viable cells decreases, being indicative of cell lysis by the replicating virus.

1. A method of determining viral infection in a subject, the method comprising:
analyzing a breath sample of said subject, wherein said analyzing comprises:
a. from said breath sample, obtaining input data specifying one or more volatile organic compound (VOCs) associated with replication of a defined virus in a host cell;
b. querying a database using said input data to identify a marker profile associated with the at least one of said specified one or more VOCs; and
c. contingent upon the identification of said marker profile in said database, providing output data comprising said marker profile and the defined virus.

2. The method of claim 1, wherein said one or more VOCs are specific to the defined virus and to a defined host cell.

3. The method of claim 1 wherein said one or more VOCs are specific to a defined virus strain.

4. (canceled)

5. The method of claim 1, wherein said database comprises viral infection clusters, each cluster defines a marker profile for a defined virus and/or a defined host cell.

6. (canceled)

7. The method of claim 1, wherein said marker profile is determined by an in vitro assay comprising inoculating at least one defined host cell in a culture media with a defined virus strain and determining presence or absence of VOCs released to the surrounding of said culture media during replication of said defined virus strain.

8. The method of claim 5, wherein said in vitro assay comprises a plurality of tests, each test comprising inoculating a differently defined host cell in a culture media with a same defined virus strain and determining presence or absence of VOCs released to the surrounding of culture media during replication of said defined virus strain that are common to said plurality of tests.

9. (canceled)

10. The method of claim 5, comprising deducing from collected VOCs, background VOCs, said background VOCs are VOCs released to surrounding of a same culture media in an in vitro assay where said host cells are cultured in said culture media without inoculation with a virus.

11. (canceled)

12. (canceled)

13. The method of claim 1 to 12, wherein said output data comprises determination of type of virus causing said viral infection in said subject and/or type of host cell infected with said virus.

14. (canceled)

15. (canceled)

16. The method of claim 1, wherein said output data comprises at least one treatment protocol against said viral infection and optionally statistical data regarding chances of success of said treatment.

17. A method of monitoring a viral infection in a subject, the method comprising:
a. obtaining from a breath sample of said subject input data specifying one or more volatile organic compounds (VOC), said sample being obtained at a determination time point;
b. querying a database using said input data to identify a marker profile associated with the at least one of said specified one or more VOC;
c. contingent upon the identification of said specified one or more VOC in said database, providing output data comprising said marker profile associated with the defined virus; and
d. comparing said output data with prior data corresponding to a marker profile obtained for said subject at a time preceding the determination time point; wherein a change in said marker profile being indicative of a viral infection state.

18. The method of claim 17, wherein said determination time point is during treatment of said subject against the viral infection and/or before initiation of treatment of said subject against the viral infection.

19. (canceled)

20. The method of claim 17, wherein said profile marker is indicative of the viral infection state.

21. The method of claim 17, wherein said output data comprises at least one treatment protocol against said viral infection.

22. A method of determining a treatment protocol for a subject being suspected of having a viral infection, the method comprising:
a. determining for said subject a viral marker profile, said determination comprises:
i. from said breath sample obtaining input data specifying one or more volatile organic compounds (VOC) associated with replication of a defined virus in a host cell;
ii. querying a database using said input data to identify said viral marker profile, the marker profile being associated in said database with a defined virus and a defined host cell; and
iii. contingent upon identification of said viral marker profile, providing output data with respect to the defined virus and the defined host cell; said output data comprises determination of type of virus causing said viral infection in said subject;
b. determining or selecting a treatment protocol for the identified virus.

23. The method of claim 22, wherein said marker profile is specific to a defined virus strain and/or to a defined virus and a defined host cell.

24. (canceled)

25. (canceled)

26. The method of claim 17, wherein said marker profile is determined by an in vitro assay comprising inoculating at least one defined host cell in a culture media with a defined virus strain and determining presence or absence of VOCs released to the surrounding of said culture media during replication of said defined virus strain.

27. The method of claim 26, wherein said in vitro assay comprises a plurality of tests, each test comprising inoculating a differently defined host cell in a culture media with a same defined virus strain and determining presence or absence of VOCs released to the surrounding of culture media during replication of said defined virus strain that are common to said plurality of tests.

28. (canceled)

29. The method of claim 26, comprising deducing from collected VOCs, background VOCs, said background VOCs are VOCs released to surrounding of a same culture media in an in vitro assay where said host cells are cultured in said culture media without inoculation with a virus.

30. (canceled)

31. (canceled)

32. A method of treatment of a subject having a viral infection, the method comprises providing said subject with an anti-viral treatment, wherein said anti-viral treatment is determined by the method of claim 22.

33. A database, comprising:
a structured collection of records including a bank of marker profiles associated with a bank of defined viruses;
wherein said viral marker profile comprises specified VOCs that are released from at least one defined host cell during viral replication with a defined virus.

34. The database of claim 33, wherein said viral marker profile comprises specified VOCs that are commonly released from two or more differently defined host cells during viral replication of a defined virus strain.

35. The database of claim 33, wherein said marker profile is pre-associated with a defined virus in an in vitro assay comprising identification of one or more VOCs from debris of at least one host cell infected with the defined virus causing said viral infection.

36. (canceled)

37. The database of claim 33, wherein each of said marker profiles comprise specified VOCs that associate between a defined virus strain and a defined host cell.

38. (canceled)

39. (canceled)

40. (canceled)

41. (canceled)
